(19) United States
(12) Patent Application Publication
(10) Pub. No.: US 2013/0110095 A1
(43) Pub. Date: May 2, 2013

(54) Method of correcting vision problems using only a photodisruption laser

(71) Applicant: Brian S. Boxer Wachler, Santa Monica, CA (US)

(72) Inventor: Brian S. Boxer Wachler, Santa Monica, CA (US)

(21) Appl. No.: 13/317,711

(22) Filed: Oct. 26, 2011

(51) Int. Cl. A61F 9/01

(52) U.S. Cl. 606/5

(57) ABSTRACT

A method for correcting myopia, hyperopia, astigmatism, and multi-focal vision problems is accomplished without the use of a microkeratome or an Excimer laser by using a photodisruption laser such as a femtosecond (“FS”) laser to form a flap of varying thicknesses and radii of curvature. This procedure enables a refractive surgeon to reshape the cornea by controlling the shrinkage of the cornea's collagen fibers.

### FIELD OF THE INVENTION

The present invention relates to an improvement over lamellar laser refractive surgery, also known as LASIK. The invention is known by the acronym C-CLEAR™, and technically it is not a form of LASIK surgery and does not require an Excimer laser. In the C-CLEAR™ surgery a photodisruption laser such as a femtosecond (“FS”) laser is used to create a flap of varying thickness at varying locations on the cornea, thus enabling corrections of myopia and hyperopia, astigmatism, and even multifocal problems. The procedure is performed entirely with a single FS laser, without the use of an Excimer laser. A photodisruption laser functions at the molecular level, which permits corrections by dissecting collagen planes of varying, predetermined thicknesses. By controlling the contraction of the cornea's collagen with varying flap thicknesses, the cornea's radius of curvature is modified at different locations on the flap to correct a patient's vision. The procedure is particularly effective for low levels of vision correction.

### BACKGROUND OF THE INVENTION

LASIK is lamellar laser refractive surgery. To begin a LASIK procedure, the surgeon creates a partial thickness lamellar corneal flap under which he performs Excimer laser ablation. While laser-based refractive surgery has become a popular and successful method of treating myopia (near-sightedness), hyperopia (far-sightedness), astigmatism, and other ophthalmic problems, certain public misperceptions still inhibit millions of people who wear glasses and contact lenses from having their vision corrected with LASIK. The single greatest public apprehension seems to be the laser itself. Many potential patients view the Excimer laser as “burning” the eye tissue, and thus a “risky” and “invasive” procedure. While the perceived risks and discomfort of a laser procedure are for the most part unjustified, ophthalmic surgeons and laser manufacturers must nevertheless contend with this misperception in convincing the many worriers and skeptics who resist giving up their glasses or contacts. In contrast, many laser surgery candidates are either unaware or undeterred by the notion that in LASIK the surgeon will create a flap in the cornea before using the Excimer laser.

The largest single group of potential laser surgery candidates includes the millions of individuals who suffer from low levels of myopia and hyperopia. The inventor's experience has been that individuals with less serious vision problems often seem to be the ones most hesitant to correct their vision with refractive laser surgery. Individuals with low level myopia and hyperopia, i.e., up to about 3.0 diopters, with modest or no astigmatism often stay with the correction provided by glasses or contact lenses, or a combination of both because of their apprehension of “the laser that burns tissue”. Public fear is over the Excimer laser component of LASIK—most people are not even aware LASIK involves creation of a flap. Conversely, those patients with higher levels of myopia and hyperopia for whom glasses or contacts do not provide a satisfactory solution, either in terms of vision correction, convenience, or appearance, tend to be more willing to try laser surgery and endure their perceived risks of LASIK. Therefore, a continuing need exists to develop tools for refractive surgery that are simpler, less invasive, and which are less daunting, especially to prospective patients with low myopia and low hyperopia.

In recent years, technological developments in laser eye surgery have focused more on the precision of the surgery than on changing laser surgery to address the perceptions of those who are disinclined to seek its benefits. For example, Wavefront technology has been touted as groundbreaking because it has the potential to improve not only how much one can see—visual acuity measured by the standard 20/20 eye chart—but also how well one can see, in terms of contrast sensitivity and fine detail. This vision improvement translates into a reduced risk of post-LASIK complications, such as glare, halos and difficulty with night vision. Nevertheless, Wavefront correction still requires the use of the Excimer laser. Another dramatic improvement has appeared in the form of the femtosecond (FS) laser. Whereas earlier versions of LASIK used microkeratomes to cut meniscus-shaped flaps of uneven thickness, the precision of the FS laser permits the surgeon to fashion precisely shaped flaps of uniform, planar thickness. As the FS laser and its planar flap secured its clinical success and its success in the marketplace, microkeratome manufacturers improved their devices so that they can now cut planar flaps precisely enough to compete with the precision of the FS laser. Therefore, as clinical practice currently stands, both the FS laser and the newer versions of the microkeratome focus on creating a uniform planar flap.

The FS laser represents the current peak of photodisruption laser technology. Unlike the Excimer laser, the FS laser uses infrared light to precisely shape a corneal flap by a process known as intrastromal photodisruption. Infrared laser pulses form a cavity inside the cornea, which is much different than the surface blade cutting of the microkeratome or the burning ablation of the Excimer laser. The FS laser focuses the beam's energy onto a tiny, 2-3 micron spot. The laser beam moves delicately through the outer layers of the cornea until it reaches the beam's exact focal point. When the beam reaches this point it forms microscopic bubbles of carbon dioxide and water vapor. Many thousands of these bubbles are placed at a precisely-controlled depth to define a dissection plane. This is the actual photodisruption process. The fast-firing FS laser creates these microscopic bubbles at a large number of locations within the cornea. It then connects these tiny bubbles under the outside surface of the cornea so that a corneal flap can be created with extraordinary precision. The focused laser pulse divides tissue at the molecular level without the transfer of heat or the impact of cutting the surrounding tissue. The FS laser offers a myriad of impressive advantages, most notably the ability to form the corneal flap into virtually any shape and size; to choose the diameter, shape, thickness, depth, angle, and location of corneal flaps and their hinges.

An FS laser generates light pulses as short as one quadrillionth of a second. In hyperopia this feature enables the FS laser to transect the trabecular meshwork in corneoscleral rim tissues with little or no collateral damage. The laser dissects tissue more efficiently, using fewer shots and less cumulative energy. Pulse energy of an FS laser is measured in μJ (microjoules), with total energy densities ranging in the area of 1 J per mm². The FS laser goes beyond simply creating a safer, more precise, planar flap. It prepares an optimal corneal architecture below the flap, thus creating superior visual outcomes. The end result: fewer complications and better vision.

Despite these improvements, such as Wavefront LASIK, the FS laser, and the improved microkeratome, the surgeon and patient are still left with only two basic options for reshaping the cornea. Each option, single-step ablation or the preliminary creation of a lamellar flap, requires the use of an Excimer laser, the “risky,” “burning,” “invasive” aspect of LASIK that inspires dread in some prospective patients, in contrast to the flap part of the LASIK procedure of which most are unaware. In the case of spherical corrections of myopia and hyperopia, increasing or decreasing the cornea's radius of curvature correctly focuses the patient's vision. This is accomplished when the Excimer laser ablates away cornea tissue. Correcting astigmatism carries the same problematic implications. The refractive error of the astigmatic eye stems from a difference in degree of curvature refraction of the two different meridians, i.e., the eye has different focal points in different planes. For example, the image may be clearly focused on the retina in the horizontal (sagittal) plane, but not in the vertical (tangential) plane. To rectify astigmatism the refractive surgeon will ablate the thicker meridian so the degree of curvature of each meridian is the same.

Excimer ablation of tissue, with or without a flap, has inherent problems. Despite continuing improvements in laser design, eye tracking, and laser algorithms, the post-operative refractive outcomes of Excimer ablation are not always as precise as the pre-operative measurements and the intra-operative laser applications would lead one to anticipate. In addition, there are common side-effects. The most common is glare or halos with night vision. Dry eyes, which can persist for several months after surgery, are also a commonly reported problem. Although uncommon, a small percentage of LASIK patients will suffer some degree of permanent dry eye, especially if the Excimer laser cuts a plethora of corneal nerves. In correcting hyperopia, most laser energy is directed at the periphery rather than at the center of the cornea. In that situation careful attention must be paid to centering and tracking, or else the ablation can induce astigmatism. Another drawback of the Excimer laser, when ablation is being used in conjunction with a corneal flap, is the expense involved with a more complicated procedure that requires a second piece of equipment, whether that equipment is the microkeratome or the FS laser. Therefore, it is desirable to have a single piece of equipment that could be used in refractive laser surgery for vision correction that could eliminate some of the clinical problems of Excimer lasers and could also circumvent or assuage the public's concern about the risk or invasiveness they associate with the use of Excimer lasers in LASIK procedures.

In ophthalmic surgery one well-known cornea property is its composition, namely collagen. Also known is that in certain circumstances collagen fibers will contract when they are cut. The Achilles tendon is one classic, non-ocular example. Tendons are composed of collagen, and when the Achilles tendon ruptures the tendons recoil like a stretched rubber band that has been instantaneously sheared. In LASIK surgery corneal flap collagen will contract if there is a difference in flap thickness. For example, contraction occurs when collagen tissues are cut by an older microkeratome, which cuts a meniscus-shaped flap. Because of its design, the older microkeratome necessarily leaves a thinner portion at the flap center than it does at the periphery of the flap. This result induced surgeons and equipment manufacturers to focus on creating a planar flap rather than the meniscus-shaped flap. It appeared to refractive surgeons that if the thickness of a corneal flap varied significantly, it would not be preferred over a planar flap with symmetrical collagen contraction. Research such as this has reinforced efforts to insure that corneal flaps are uniform and planar rather than meniscus-shaped.

Despite the FS laser's remarkable precision at the molecular level, its lack of ablative effect, and the improvement that the laser has given to LASIK outcomes, FS laser manufacturers have touted their product solely for replacing the microkeratome to create a uniform flap. While the FS laser presents surgeons with a vast array of parameters for creating the flap, the computer that accompanies the laser limits the surgeon, because the computer program controls parameters that only result in a uniformly thick, planar flap. Therefore, it is desirable to have a laser for refractive surgery that represents an alternative to the Excimer laser-based LASIK—one that is likely to appeal to the millions of people suffering from low myopia and low hyperopia; that is less expensive; that is less likely to have adverse effects such as glare, halos, and dry eye; that is less invasive and promotes quicker healing; that is more accurate; that is less costly; and, whose effects and techniques are less intimidating to the public.

### SUMMARY OF THE INVENTION

The present invention solves these problems by creating a refractive flap to correct vision without the use of an Excimer laser. Using the FS laser to create the flap has none of the burning and pain that the public associates with an Excimer laser. In addition, the nature of the invention can be explained in terms that can alleviate the fears of many who are afraid to try LASIK surgery. The creation of microscopic bubbles and even the term photodisruption sound far less intimidating than the searing notion of an Excimer laser “burning” or “ablating” corneal tissue from the public's perception. Unlike the current focus on flap technology, the method of the present invention requires the creation of a non-planar flap with varying thicknesses at different locations on the flap while avoiding the use of the Excimer laser.

As noted above, the present invention goes by the acronym of C-CLEAR™ and is a procedure that uses only the FS laser to create a corneal flap of uneven thickness, which results in the contraction of the collagen in the cornea. The contraction of the collagen, in turn, alters the cornea's radius of curvature. Depending on the varying thicknesses of the corneal flap, the radius of curvature is altered to correct for myopia, hyperopia, astigmatism, and even multi-focal vision problems. Because of the apparent physical limitations caused by collagen contraction, cornea thickness, and a safe minimal flap thickness, it is anticipated that this method will be more effective for relative low levels of vision correction. This would include, for example, myopia and hyperopia of up to 3.00 OD. The claims of the invention, however, should not be considered so limited, because future developments could extend the benefits of the procedure beyond what is considered low-level vision correction.

C-CLEAR™ is a method of contracting a cornea's collagen fibers with a photodisruption laser to make visual corrections by dissecting a corneal flap, comprising the steps of: creating and analyzing a set of vision parameters associated with a patient's eye, the parameters including a vision correction for the eye and thicknesses and radii of curvature of a multiplicity of points of the eye's cornea, the multiplicity of points further including a plurality of points at the center of the flap and a plurality of points at the periphery of the flap; entering the vision parameters into a computer, wherein the computer is associated with a photodisruption laser; analyzing the vision parameters with the computer to determine a flap thickness at each of the multiplicity of points; and, using the photodisruption laser to form a flap on the cornea, wherein the flap has a multiplicity of thicknesses and radii of curvature in the vicinity of the center and in the periphery of the flap that correlate to the shrinkage of the collagen fibers in the flap and to the visual correction.

In another embodiment C-CLEAR™ represents an improved method of making a low-level vision correction with laser refractive surgery that excludes the use of an excimer laser, the improvement comprising the steps of programming a photodisruption laser system with a plurality of thicknesses and radii of curvature of a cornea; and, forming a flap on the cornea with the photodisruption laser, wherein the flap has a multiplicity of thicknesses and radii of curvature that correspond to the vision correction.

Additional embodiments are described and claimed below.

### BRIEF DESCRIPTION OF THE DRAWINGS

Below is a detailed description that refers to the novel aspects of C-CLEAR™, a variety of structural equivalents known by those of skill in the art, and in that context refers to the following figures.

FIG. 1 is perspective drawing of an eye with a corneal flap.

FIG. 2 is a chart that plots the difference in flap thickness between the center and periphery of a flap against the optical correction in diopters.

FIG. 3 is a cross-section of an exemplary flap as it would be formed to correct low level myopia.

FIG. 4 is a cross-section of an exemplary flap as it would be formed to correct low level hyperopia.

FIG. 5 is a topographic plot of a myopic eye.

FIG. 6 is a topographic plot of an astigmatic eye.

### DETAILED DESCRIPTION OF THE INVENTION

C-CLEAR™ is based on the nature of the corneal stroma and its reaction to being transected. The stroma is composed of about 200 flattened lamellae (plates of collagen fibrils), superimposed one on another. Each is about 1.5-2.5 micrometers thick. The fibers of each lamella are parallel with one another, but at right angles to those of adjacent lamellae. These lamellae are made up of bundles of modified connective tissue, the fibers of which are directly continuous with those of the sclera. Fibers of the layers frequently interweave. The collagen fibrils run at different angles between points on the corneal limbus, the border of the cornea, and the sclera.

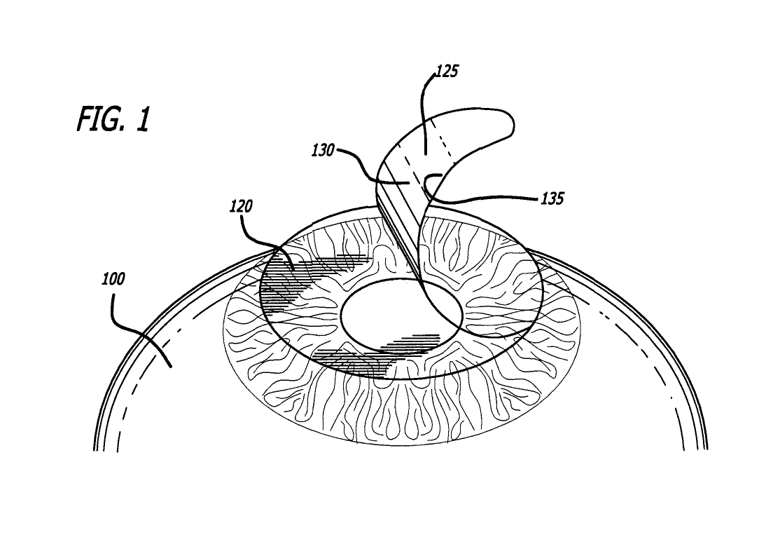

FIG. 1 depicts an eye 100 with a cornea 120 from which a corneal flap 125 has been formed and folded back. The flap has a center 130 and an edge or periphery 135. Let Δt be the difference in flap thickness between the flap center 130 and the flap edge 135. Expressed algebraically, Δt=t_{periphery}−t_{center}. In current LASIK surgery an FS laser uses photodisruption to form a uniformly thick the flap. In other words, Δt=0, regardless of the procedure. In C-CLEAR™, Δt does not equal zero. To correct myopia the flap is thinner in the center than the periphery, so Δt is greater than zero. Conversely, for hyperopia Δt is less than zero. C-CLEAR™ is not, however, limited to a uniform Δt at the periphery or in the area between the center and periphery of the flap. For multifocal or asymmetric corrections, the flap thickness at the edge 135 and between the edge 135 and center 130 can be varied because of the FS laser's precision. A flap with different thicknesses at the periphery is programmed into the FS laser's computer and the laser accomplishes the necessary flap creation. Neither a microkeratome nor an excimer laser is necessary. Once the flap 125 is formed, the surgeon should fold it back as he would if he were performing standard LASIK surgery with an Excimer laser. Because the flap's collagen fibrils contract immediately, the surgeon can replace the flap without waiting.

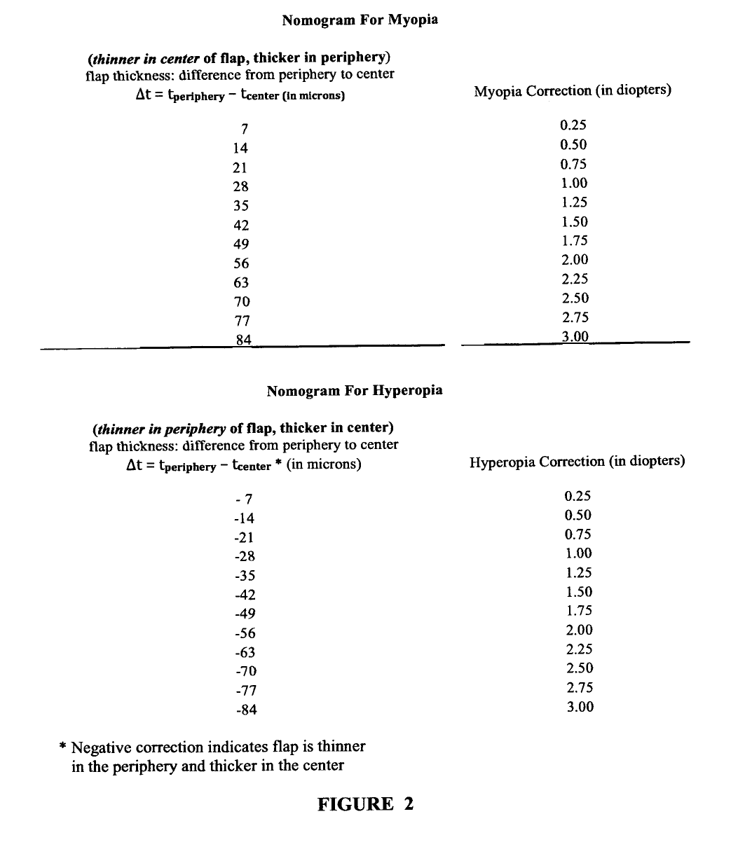

FIG. 2 charts monofocal corrections for simple myopia and hyperopia. The Δt represents the difference between the center and periphery of the flap. In this particular example, the thicknesses of the intervening points of the flap can be determined by using a linear ratio based on their relative distance between the center and periphery. For corneas that are not uniformly thick or that have a slightly uneven surface, the Δt can also be adjusted for the relative thickness of the flap at any given point.

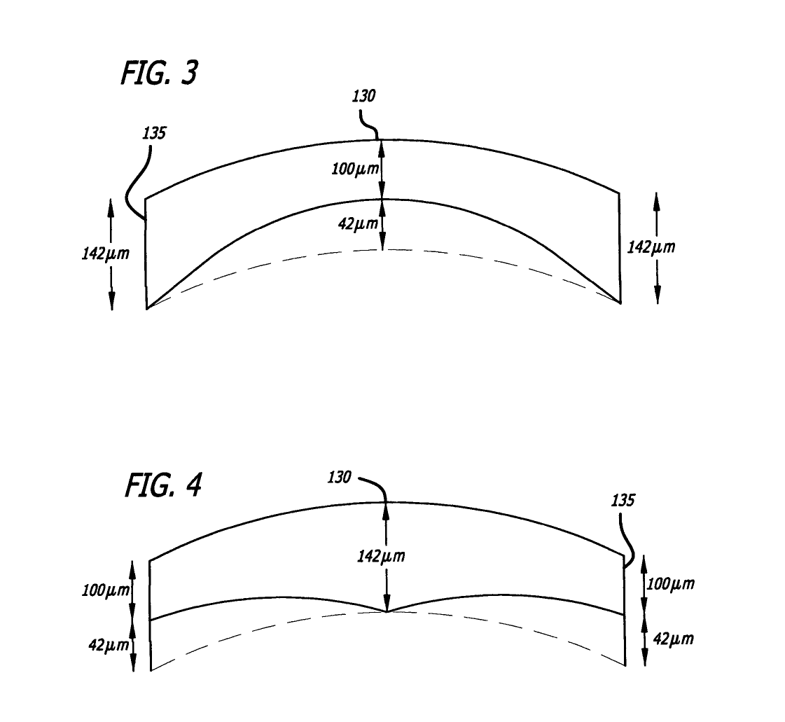

An example of the preceding discussion can be seen in FIG. 3 and FIG. 4. FIG. 3 represents a flap cross-section with flap center 130 and flap edge 135. Assume that a correction of 1.50 OD is necessary to correct a patient's low level myopia. Also assume a minimum flap thickness of 100 μm. The FS laser is programmed to form the flap center 130 to be 100 μm thick and the flap edge to be 142 μm thick. Δt=142 μm−100 μm=+42 μm, which corresponds to a 1.50 OD correction in FIG.2. For ease of comparison FIG. 4 uses the same numbers as FIG. 3, but the flap configuration is reversed. Δt=100 μm−142 μm=−42 μm. The negative result indicates that the correction is for hyperopia.

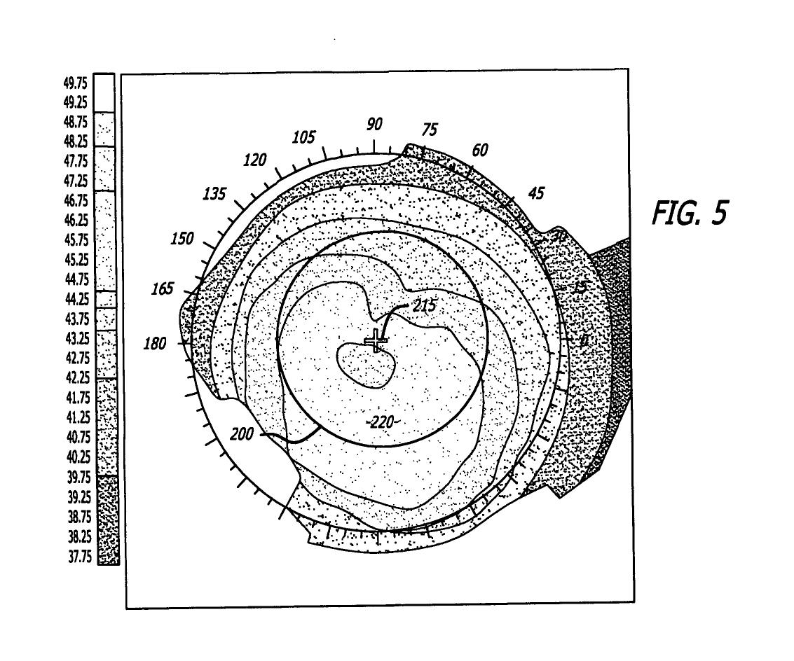

FIG. 5 is a corneal topographic map of an eye with mild myopia of −1.00 spherical diopters and no appreciable astigmatism. The eye's center 215 is indicated by a “+”. Circle 200 indicates the edge of the pupil. The flap that would be formed with the photodisruption laser would be well outside the pupil, but is not shown in FIG. 5. The numerical and shaded scale 225 on the left represents the curvature of the cornea in diopters. The more lightly shaded area 220 of the cornea, both inside and outside the lower half of pupil area 200 requires slight flattening to correct the myopia. As indicated on the chart in FIG. 2, the Δt would be 28 μm. This limits the number of collagen fibrils that will automatically shrink, and therefore limits the change in the cornea's radius of curvature. The total flap thickness, including the Δt of 28 μm, will be determined by the nature of the entire cornea and the necessary size of the flap.

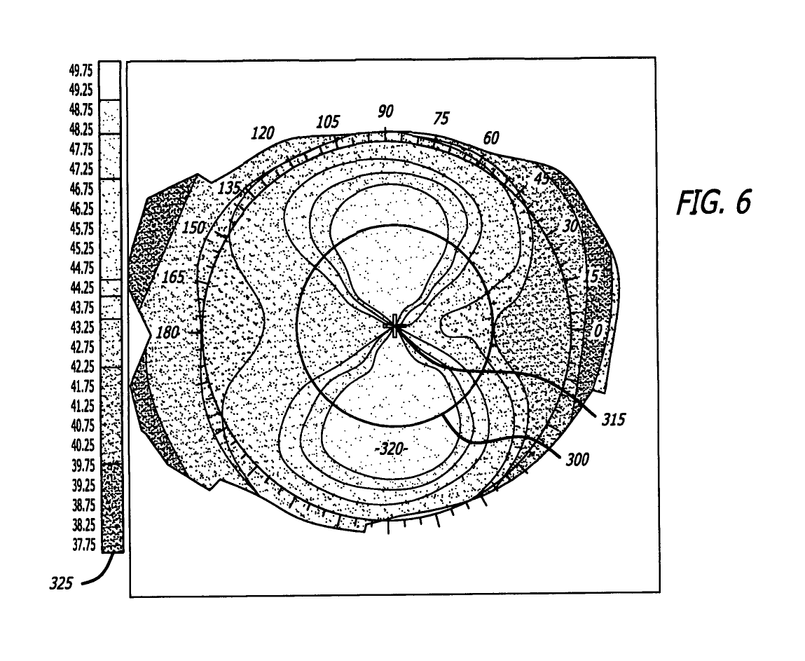

FIG. 6 is a corneal topographic map of a −1.00 D myopic eye with astigmatism of 1.75 D cylindrical diopters. The pupil's edge is indicated by circle 300. The center of the cornea and pupil 315 is again indicated by “+” and the scale 325 at left is the radius of curvature of the cornea in diopters. The flap that would be created with the photodisruption laser would be well outside the pupil, but is not shown in FIG. 6. The more lightly shaded areas 325, above and below the eye's center 315, represent a steep meridian, while the more darkly shaded areas 335 on the right and left of center 315 represent a flatter meridian. The appropriate treatment would be to flatten the steep portion of the cornea 320 that is causing the astigmatism, which would also induce a specified amount of overall flattening and correct the myopia. This requires that the flap periphery be thicker than the flap center in the steeper portions of the flap, which would encompass the more lightly shaded areas 325. The more darkly shaded areas 335 to the right and left of the center of the eye would have the same thickness at the flap periphery as at the flap center, so that the cornea collagen fibrils would not shrink.

Although the inventor has described what he considers the best mode of carrying out the invention, it will be apparent to those skilled in the art that modifications, variations, and equivalents can be made without departing from the scope of the invention as detailed in the claims below. For example, it is anticipated that technological improvements could result in the replacement of the FS laser with another photodisruption device or another form of device that does not ablate tissue like the Excimer laser. Similarly, it should be understood that the flap thicknesses specified in the nomograms in FIG. 2 may vary, depending upon the clinical setting, the patient's individual vision parameters, and the instrument used to create the flap.

What is claimed is:

1. A method of shrinking a cornea's collagen fibers with a photodisruption laser to make visual corrections by creating a corneal flap, comprising the steps of:
creating and analyzing a set of vision parameters associated with a patient's eye, the parameters including a desired vision correction for the eye and thicknesses and radii of curvature of a multiplicity of points of the eye's cornea, the multiplicity of points further including a plurality of points at the center of the flap and a plurality of points at the periphery of the flap;
entering the vision parameters into a computer, wherein the computer is associated with a photodisruption laser;
analyzing the vision parameters with the computer to determine a flap thickness at each of the multiplicity of points; and,
using the photodisruption laser to form a flap on the cornea, wherein the flap has a multiplicity of thicknesses and radii of curvature in the vicinity of the center and periphery of the flap that correlate to the shrinkage of the collagen fibers in the flap and to the visual correction.

2. The method of claim 1, wherein the photodisruption laser is a femtosecond laser.

3. The method of claim 1, further comprising the step of folding the flap back from the cornea.

4. The method of claim 3, further comprising the step of replacing the flap on the cornea.

5. The method of claim 1, wherein the vision parameters further include an additional multiplicity of points between the center and the periphery of the flap whose thicknesses are based on their relative distances from the center and the periphery of the flap.

6. The method of claim 5, wherein the vision parameters further include an analysis of the total cornea thickness at the center, at the periphery, and at the additional multiplicity of points in between, and wherein the analysis results in a plurality of different flap thicknesses at the center, periphery, and points in between that correspond to the desired vision correction.

7. An improved method of making a low-level vision correction with laser refractive surgery that excludes the use of an excimer laser, the improvement comprising the steps of
programming a photodisruption laser system with a plurality of thicknesses and radii of curvature of a cornea; and,
forming a flap on the cornea with the photodisruption laser, wherein the flap has a multiplicity of thicknesses and radii of curvature that correspond to the vision correction.

8. The improved method of claim 7, further comprising the step of folding back the flap back from the cornea.

9. The improved method of claim 8, further comprising the step of replacing the flap on the cornea.

10. The improved method of claim 7, wherein the photodisruption laser is a femtosecond laser.
